(19) United States
(12) Patent Application Publication
(10) Pub. No.: US 2017/0246265 A1
(43) Pub. Date: Aug. 31, 2017

(54) PHARMACEUTICAL PREPARATIONS FOR ATTENTION DEFICIT DISORDER, ATTENTION DEFICIT HYPERACTIVITY DISORDER AND OTHER ASSOCIATED DISORDERS

(71) Applicant: Curemark, LLC, Rye, NY (US)

(72) Inventor: Joan M. Fallon, Bronxville, NY (US)

(21) Appl. No.: 15/354,940

(22) Filed: Nov. 17, 2016

Related U.S. Application Data
(62) Division of application No. 11/533,818, filed on Sep. 21, 2006, now abandoned.
(60) Provisional application No. 60/744,922, filed on Apr. 15, 2006.
(60) Provisional application No. 60/744,928, filed on Apr. 15, 2006.
(60) Provisional application No. 60/719,028, filed on Sep. 21, 2005.
(60) Provisional application No. 60/719,255, filed on Sep. 21, 2005.

(51) Int. Cl. A61K 38/48; A61K 31/137; A61K 38/47; A61K 38/46; A61K 9/20; A61K 47/38; A61K 31/138

(52) U.S. Cl. CPC A61K 38/4826; A61K 47/38; A61K 31/137; A61K 31/138; A61K 38/465; A61K 9/2095; C12Y 304/21001; A61K 38/47; A61K 38/48

(57) ABSTRACT

A pharmaceutical preparation for the treatment of attention deficit disorders combines a therapeutically effective amount of digestive enzymes, such as chymotrypsin, and medication used to treat attention deficit disorders, such as Ritalin®, Concerta®, Adderall® and Strattera®. The preparation may be in the form of a tablet, capsule or time released formula in order to reduce the amount of pills per dosage. The pharmaceutical preparation ameliorates the symptoms of the attention deficit disorder. The preparation has a stabilizing matrix containing a solidified microcrystalline cellulose which captures and protects therapeutically effective amounts of digestive enzyme particles within the stabilizing matrix.

### RELATED APPLICATIONS

This application claims the benefit of U.S. Provisional Application Nos. 60/719,028, filed Sep. 21, 2005, 60/719,255 filed Sep. 21, 2005, 60/744,922 filed Apr. 15, 2006, and 60/744,928 filed Apr. 15, 2006. These applications are hereby incorporated by reference in their entirety.

### FIELD OF THE INVENTION

The present invention is directed to therapeutic agents for the treatment of attention deficit disorder, attention deficit hyperactivity disorder, and other associated disorders. More specifically, the present invention relates to pharmaceutical preparations containing, but not limited to, methylphenidate, methylphenidate salts, amphetamines, amphetamine salts, atomoxetine HCl and digestive and/or pancreatic enzymes including, but not limited to, amylases, proteases, cellulase, papaya, bromelain, lipases, chymotrypsin, trypsin, carboxypeptidase, elastase; hydrolase, pancreatin and pancrelipase. This combination is made either by direct compression, wet granulation or other methods including, but not limited to, the use of Prosolv technology, time-release technology and lipid encapsulation.

### BACKGROUND OF THE INVENTION

Attention Deficit Disorder and Attention Deficit Hyperactivity Disorder (ADD/ADHD) is a condition that becomes apparent in some children in the preschool and early school years. It is hard for these children to control their behavior, pay attention, and attend to tasks. It is estimated that between 3 and 5 percent of children have ADD/ADHD, or approximately 2 million children in the United States. This means that in a classroom of 25 to 30 children, it is likely that at least one will have ADD/ADHD.

The principal characteristics of ADD/ADHD are inattention, hyperactivity, and impulsivity. These symptoms appear early in a child's life. Symptoms of ADD/ADHD will appear over the course of many months, often with the symptoms of impulsiveness and hyperactivity preceding those of inattention, which may not emerge for a year or more. Different symptoms may appear in different settings, depending on the demands the situation may pose for the child's self-control. According to the most recent version of the Diagnostic and Statistical Manual of Mental Disorders (DSM-IV-TR), there are three patterns of behavior that indicate ADD/ADHD. People with ADD/ADHD may show several signs of being consistently inattentive. They may have a pattern of being hyperactive and impulsive far more than others of their age. Or they may show all three types of behavior. This means that there are three subtypes of ADD/ADHD recognized by professionals. These are the predominantly hyperactive-impulsive type (that does not show significant inattention); the predominantly inattentive type (that does not show significant hyperactive-impulsive behavior); and the combined type (that displays both inattentive and hyperactive-impulsive symptoms).

Over the last few decades, scientists have come up with possible theories about what causes ADD/ADHD, including environmental factors, brain injury, food additives and sugar, and genetics.

Studies have shown a possible correlation between the use of cigarettes and alcohol during pregnancy and risk for ADD/ADHD in the offspring of that pregnancy. As a precaution, it is best during pregnancy to refrain from both cigarette and alcohol use. Another environmental agent that may be associated with a higher risk of ADD/ADHD is high levels of lead in the bodies of young preschool children. Since lead is no longer allowed in paint and is usually found only in older buildings, exposure to toxic levels is not as prevalent as it once was. Children who live in old buildings in which lead still exists in the plumbing or in lead paint that has been painted over may be at risk.

One early theory was that attention disorders were caused by brain injury. Some children who have suffered accidents leading to brain injury may show some signs of behavior similar to that of ADD/ADHD, but only a small percentage of children with ADD/ADHD have been found to have suffered a traumatic brain injury.

It has been suggested that attention disorders are caused by refined sugar or food additives, or that symptoms of ADD/ADHD are exacerbated by sugar or food additives. In 1982, the National Institutes of Health held a scientific consensus conference to discuss this issue. It was found that diet restrictions helped about 5 percent of children with ADD/ADHD, mostly young children who had food allergies. A more recent study on the effect of sugar on children, using sugar one day and a sugar substitute on alternate days, without parents, staff, or children knowing which substance was being used, showed no significant effects of the sugar on behavior or learning. In another study, children whose mothers felt they were sugar-sensitive were given aspartame as a substitute for sugar. Half the mothers were told their children were given sugar, half that their children were given aspartame. The mothers who thought their children had received sugar rated them as more hyperactive than the other children and were more critical of their behavior.

Attention disorders often run in families, so there are likely to be genetic influences. Studies indicate that 25 percent of the close relatives in the families of ADD/ADHD children also have ADD/ADHD, whereas the rate is about 5 percent in the general population. Many studies of twins now show that a strong genetic influence exists in the disorder.

Some knowledge of the structure of the brain is helpful in understanding the research scientists are doing in searching for a physical basis for attention deficit hyperactivity disorder. One part of the brain that scientists have focused on in their search is the frontal lobes of the cerebrum. The frontal lobes allow us to solve problems, plan ahead, understand the behavior of others, and restrain our impulses. The two frontal lobes, the right and the left, communicate with each other through the corpus callosum, (nerve fibers that connect the right and left frontal lobes). The basal ganglia are the interconnected gray masses deep in the cerebral hemisphere that serve as the connection between the cerebrum and the cerebellum and, with the cerebellum, are responsible for motor coordination. The cerebellum is divided into three parts. The middle part is called the vermis. All of these parts of the brain have been studied through the use of various methods for seeing into or imaging the brain. These methods include functional magnetic resonance imaging (fMRI) positron emission tomography (PET), and single photon emission computed tomography (SPECT). The main or central psychological deficits in those with ADD/ADHD have been linked through these studies. By 2002 the researchers in the NIMH Child Psychiatry Branch had studied 152 boys and girls with ADD/ADHD, matched with 139 age- and gender-matched controls without ADD/ADHD. The children were scanned at least twice, some as many as four times over a decade. As a group, the ADD/ADHD children showed 3-4 percent smaller brain volumes in all regions—the frontal lobes, temporal gray matter, caudate nucleus, and cerebellum. This study also showed that the ADD/ADHD children who were on medication had a white matter volume that did not differ from that of controls. Those never-medicated patients had an abnormally small volume of white matter. The white matter consists of fibers that establish long-distance connections between brain regions. It normally thickens as a child grows older and the brain matures;

For decades, medications have been used to treat the symptoms of ADD/ADHD. The medications that seem to be the most effective are a class of drugs known as stimulants, including Ritalin® (methylphenidate), Concerta® (methylphenidate—long acting), Adderall® (amphetamine). A non-stimulant medication, Strattera® (atomoxetine HCl) has also shown promise. Strattera®, or atomoxetine HCl, works on the neurotransmitter norepinephrine, whereas the stimulants primarily work on dopamine. Both of these neurotransmitters are believed to play a role in ADD/ADHD.

It has been shown that presently marketed pharmaceutical preparations containing digestive/pancreatic enzymes are known to exhibit deficiencies with regard to content uniformity, stability and shelf life. In April of 2004 the US Food and Drug Administration issued a guideline as to the filing of new drug applications for these preparations as the presently marketed preparations of the digestive/pancreatic enzyme formulations were deemed inadequate. More specifically, digestive/pancreatic enzymes can degrade rapidly under conditions of high humidity or in the presence of other moisture sources, under light and under conditions of high temperature, and extremes in pH. Moreover, digestive enzymes are known to degrade certain pharmaceutical excipients such as carbohydrates, including lactose, sucrose, dextrose and starch, as well as certain dyes, making the current compounds on the market substandard and potentially under-medicating those who need the enzymes.

### SUMMARY OF THE INVENTION

It is a goal of the present invention to provide a stable preparation of digestive enzymes and medications used to treat attention deficit disorders which can be readily formed into a dosage formulation.

In one embodiment, the dosage formulation is administered by an oral preparation including, but not limited to, a tablet, microcapsule, minicapsule, time released capsule, sprinkle, or other methodology.

Another goal of the invention is to provide a stable pharmaceutical preparation that resists degradation by light, heat, humidity or association with commonly used excipients.

A further goal of the invention is to provide a pharmaceutical preparation in which an excipient provides a matrix to capture and protect the product before delivery.

Another goal of the invention is to provide a pharmaceutical preparation whereby the individual taking the preparation has a reduction in the amount of ADD/ADHD medication taken.

An additional goal of the invention is provide a pharmaceutical preparation whereby the individual taking the preparation has a reduction in ADD/ADHD symptomology, such as lack of attention span, hyperactivity, and impulsiveness, and/or a reduction in the secondary symptoms of ADD/ADHD, such as gastrointestinal disorders, constipation, decreased appetite, and insomnia.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a graph showing the decrease in methylphenidate dosages required after administration of a combination of methylphenidate and digestive enzymes to a group of test subjects over a period of six months.

FIG. 2 is another graph showing the decrease in methylphenidate dosages required after administration of a combination of methylphenidate and digestive enzymes to another group of test subjects over a period of four months.

FIG. 3 is a graph showing the decrease in Adderall® dosages required after administration of a combination of amphetamine and digestive enzymes to a group of test subjects over a period of six months.

FIG. 4 is a graph showing the decrease in atomoxetine HCl dosages required after administration of a combination of atomoxetine HCl and digestive enzymes to a group of test subjects over a period of six months.

FIG. 5 is a chart showing the increase in fecal chymotrypsin levels, the increase in attention span, and decrease in hyperactivity after administration of a combination of methylphenidate and digestive enzymes to a group of test subjects over a period of six months.

FIG. 6 is a chart showing the increase in fecal chymotrypsin levels, the increase in attention span, and decrease in hyperactivity after administration of a combination of methylphenidate HCl and digestive enzymes to a group of test subjects over a period of six months.

FIG. 7 is a chart showing the increase in range of foods, the decrease in constipation, and decrease in insomnia after administration of a combination of methylphenidate and digestive enzymes to a group of test subjects over a period of six months.

### DETAILED DESCRIPTION OF THE PREFERRED EMBODIMENT

Methylphenidate (Ritalin®) is a mild central nervous system (CNS) stimulant, available as tablets of 5, 10, and 20 mg for oral administration. Methylphenidate is methyl α-phenyl-2-piperidineacetate hydrochloride, as shown below:

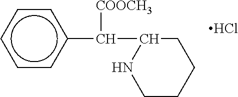

Methylphenidate hydrochloride (Concerta®) is a central nervous system (CNS) stimulant, available in four tablet strengths. Each extended-release tablet for, once-a-day oral administration contains 18, 27, 36, or 54 mg of methylphenidate HCl and is designed to have a 12-hour duration of effect. Chemically, methylphenidate HCl is d,l (racemic) methyl α-phenyl-2-piperidineacetate hydrochloride. Its empirical formula is C₁₄H₁₉NO₂.HCl. Its structural formula is:

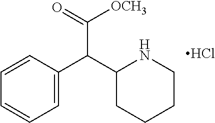 [SMILES: COC(=O)C(c1ccccc1)C1CCCCN1 ; Cl]

Adderall® is a stimulant containing amphetamine. Specifically, it combines the neutral sulfate salts of dextroamphetamine and amphetamine, with the dextro isomer of amphetamine saccharate and d,l-amphetamine aspartate monohydrate. Its structural formula is:

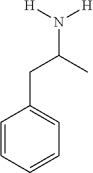

Atomoxetine HCl (Strattera®) is a selective norepinephrine reuptake inhibitor. Atomoxetine HCl is the R(−) isomer as determined by x-ray diffraction. The chemical designation is (−)-N-Methyl-3-phenyl-3-(o-tolyloxy)-propylamine hydrochloride. The molecular formula is C₁₇H₂₁NO.HCl, which corresponds to a molecular weight of 291.82. The chemical structure is:

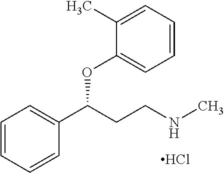 [SMILES: CNCC[C@@H](Oc1ccccc1C)c1ccccc1 ; Cl]

Atomoxetine HCl strengthens the chemical signal between those nerves that use norepinephrine to send messages. Atomoxetine HCl does not appear to affect the dopamine systems as directly as do the stimulants. Atomoxetine HCl does not seem to cause an increase in brain dopamine levels in the nucleus accumbens or the striatum areas of the brain. The stimulants appear to cause an increase in the availability of dopamine in these areas. The effect on the nucleus accumbens is believed to cause euphoria and to be responsible for the stimulants' abuse liability. Dopamine increases in the striatum may be associated with the risk of motor tics. Although the direct effect of atomoxetine HCl only seems to be with norepinephrine, it appears to cause a secondary increase in dopamine levels in the prefrontal cortex area of the brain. (the brain area behind the eyes.) This part of the brain is associated with the ability to mentally rehearse responses, and inhibit impulsivity. The area is also associated with working memory.

A stable preparation of digestive/pancreatic enzymes and ADD/ADHD medications is formed into a dosage formulation containing 1 to 100 mg of the ADD/ADHD medication and a therapeutically effective amount of a protease, an amylase, and/or a lipase. The dosage formulation may be administered by an oral preparation including, but not limited to, a tablet, mini-tabs, microcapsule, mini-capsule, time released capsule, sprinkle or other methodology.

In an embodiment utilizing methylphenidate, the dosage formulations may be as follows:

### Digestive Enzyme/Pancreatic Enzyme Combination+1 mg Methylphenidate

### Digestive Enzyme/Pancreatic Enzyme Combination+2.5 mg Methylphenidate

### Digestive Enzyme/Pancreatic Enzyme Combination+4 mg Methylphenidate

### Digestive Enzyme/Pancreatic Enzyme Combination+6 mg Methylphenidate

### Digestive Enzyme/Pancreatic Enzyme Combination+12 mg Methylphenidate

### Digestive Enzyme/Pancreatic Enzyme Combination+15 mg Methylphenidate

### Digestive Enzyme/Pancreatic Enzyme Combination+18 mg Methylphenidate

### Digestive Enzyme/Pancreatic Enzyme Combination+22 mg Methylphenidate

### Digestive Enzyme/Pancreatic Enzyme Combination+30 mg Methylphenidate

In an embodiment utilizing methylphenidate hydrochloride, the dosage formulations may be as follows:

### Digestive Enzyme/Pancreatic Enzyme Combination+2.5 mg Methylphenidate HCL

### Digestive Enzyme/Pancreatic Enzyme Combination+4 mg Methylphenidate HCL

### Digestive Enzyme/Pancreatic Enzyme Combination+9 mg Methylphenidate HCl

### Digestive Enzyme/Pancreatic Enzyme Combination+12 mg Methylphenidate HCL

### Digestive Enzyme/Pancreatic Enzyme Combination+18 mg Methylphenidate HCL

### Digestive Enzyme/Pancreatic Enzyme Combination+22 mg Methylphenidate HCl

### Digestive Enzyme/Pancreatic Enzyme Combination+32 mg Methylphenidate HCl

In an embodiment utilizing Adderall®, the dosage formulations may be as follows:

### Digestive Enzyme/Pancreatic Enzyme Combination+1.25 mg Dextroamphetamine

### Saccharate+1.25 mg Amphetamine Aspartate Monohydrate+1.25 mg Dextroamphetamine

### Sulfate+1.25 mg Amphetamine Sulfate

Digestive Enzyme/Pancreatic. Enzyme Combination+2.5 mg Dextroamphetamine

### Saccharate+2.5 mg Amphetamine Aspartate Monohydrate+2.5 mg Dextroamphetamine

### Sulfate+2.5 mg Amphetamine Sulfate

### Digestive Enzyme/Pancreatic Enzyme Combination+3.75 mg Dextroamphetamine

### Saccharate+3.75 mg Amphetamine Aspartate Monohydrate+3.75 mg Dextroamphetamine

### Sulfate+3.75 mg Amphetamine Sulfate

### Digestive Enzyme/Pancreatic Enzyme Combination+5.0 mg Dextroamphetamine

Saccharate+5.0 mg Amphetamine Aspartate. Monohydrate+5.0 mg Dextroamphetamine

### Sulfate+5.0 mg Amphetamine Sulfate

### Digestive Enzyme/Pancreatic Enzyme Combination+6.25 mg Dextroamphetamine

### Saccharate+6.25 mg Amphetamine Aspartate Monohydrate+6.25 mg Dextroamphetamine

### Sulfate+6.25 mg Amphetamine Sulfate

### Digestive Enzyme/Pancreatic Enzyme Combination+7.5 mg Dextroamphetamine

### Saccharate+7.5 mg Amphetamine Aspartate Monohydrate+7.5 mg Dextroamphetamine

### Sulfate+7.5 mg Amphetamine Sulfate

The equivalent amounts of amphetamine are 3.1 mg, 6.3 mg, 9.4 mg, 12.5 mg, 15.6 mg, and 18.8 mg respectively.

In an embodiment utilizing atomexetine HCl, the dosage formulations may be as follows:

Digestive Enzyme/Pancreatic Enzyme Combination+5 mg atomoxetine HCl
Digestive Enzyme/Pancreatic Enzyme Combination+10 mg atomoxetine HCl
Digestive Enzyme/Pancreatic Enzyme Combination+18 mg atomoxetine HCl
Digestive Enzyme/Pancreatic Enzyme Combination+0.20 mg atomoxetine HCl
Digestive Enzyme/Pancreatic Enzyme Combination+25 mg atomoxetine HCl
Digestive Enzyme/Pancreatic Enzyme Combination+30 mg atomoxetine HCl
Digestive Enzyme/Pancreatic Enzyme Combination+40 mg atomoxetine HCl
Digestive Enzyme/Pancreatic Enzyme Combination+50 mg atomoxetine HCl
Digestive Enzyme/Pancreatic Enzyme Combination+60 mg atomoxetine HCl
Digestive Enzyme/Pancreatic Enzyme Combination+70 mg atomoxetine HCl
Digestive Enzyme/Pancreatic Enzyme Combination+80 mg atomoxetine HCl
Digestive Enzyme/Pancreatic Enzyme Combination+100 mg atomoxetine HCl

The formulation resists degradation by light, heat, humidity or association with commonly used excipients. In one embodiment, an excipient provides a matrix to capture and protect the product before delivery. The stabilizing matrix consists of, but is not limited to, a solidified microcrystalline cellulose which captures and protects the therapeutically effective amounts of digestive enzyme particles. This is done through the use of Prosolv technology.

Prosolv is a combination of excipients which allow for optimized flow, compaction and product uniformity. This technology allows for uniformity in this combination, as well as manufacturing a very small tablet which would be amenable for children. With Prosolv technology, the ingredients are not just blended, but are co-processed, which assures that equal particles are uniformly distributed and these results are easily reproducible. This allows for stability and superb product quality.

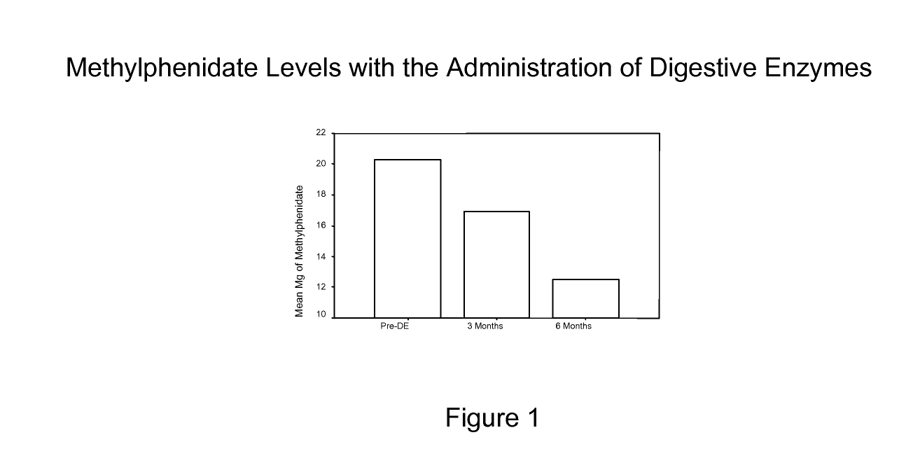

It has been shown that individuals taking the pharmaceutical preparation of the present invention have experienced a reduction in the number of capsules/tablets required per dosage. In a study conducted by the inventor, eighteen individuals diagnosed with ADD or ADHD and being treated with methylphenidate (Ritalin®) were examined. The subjects ranged in age from 6 to 15. Referring to FIG. 1, the subjects were taking an average of 20 mg of methylphenidate prior to any treatment with the present invention. After 3 months of treatment with the present invention, the subjects were taking an average of 17 mg of methylphenidate. After 6 months of treatment with the present invention, the subjects were taking 13 mg of methylphenidate. The results were similar for a group of 11 subjects being treated with methylphenidate hydrochloride (Concerta®).

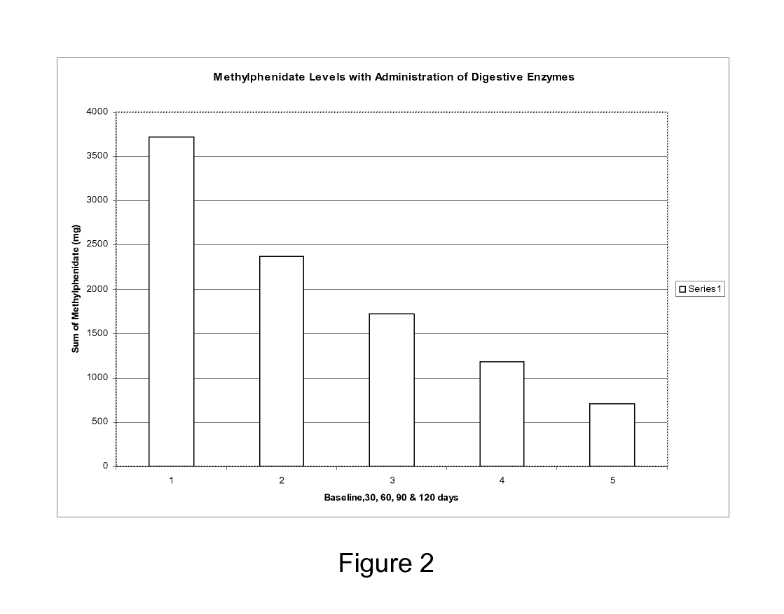

In another study, 135 subjects diagnosed with ADD or ADHD and being treated with methylphenidate (Ritalin®) were examined. Referring to FIG. 2, the subjects were taking a sum total of 3720 mg of methylphenidate prior to any treatment with the present invention. The subjects were treated with the present invention and monitored at 30, 60, 90 and 120 days. At 30 days, the sum total of methylphenidate being taken by the subjects had decreased to 2370 mg. At 60 days, the sum total of methylphenidate being taken by the subjects had decreased to 1720 mg. At 90 days, the sum total of methylphenidate being taken by the subjects had decreased to 1180 mg. At 120 days, the sum total of methylphenidate being taken by the subjects had decreased to 710 mg.

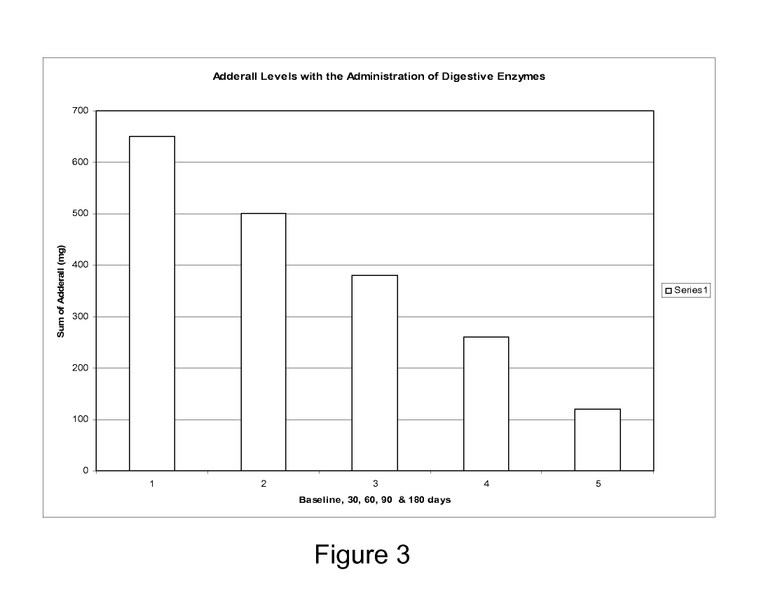

In an additional study, 33 subjects diagnosed with ADD or ADHD and being treated with Adderall® were examined. Referring to FIG. 3, the subjects were taking a sum total of 650 mg of Adderall® prior to any treatment with the present invention. The subjects were treated with the present invention and monitored at 30, 60, 90 and 180 days. At 30 days, the sum total of methylphenidate being taken by the subjects had decreased to 2370 mg. At 60 days, the sum total of methylphenidate being taken by the subjects had decreased to 1720 mg. At 90 days, the sum total of methylphenidate being taken by the subjects had decreased to 1180 mg. At 180 days, the sum total of methylphenidate being taken by the subjects had decreased to 710 mg.

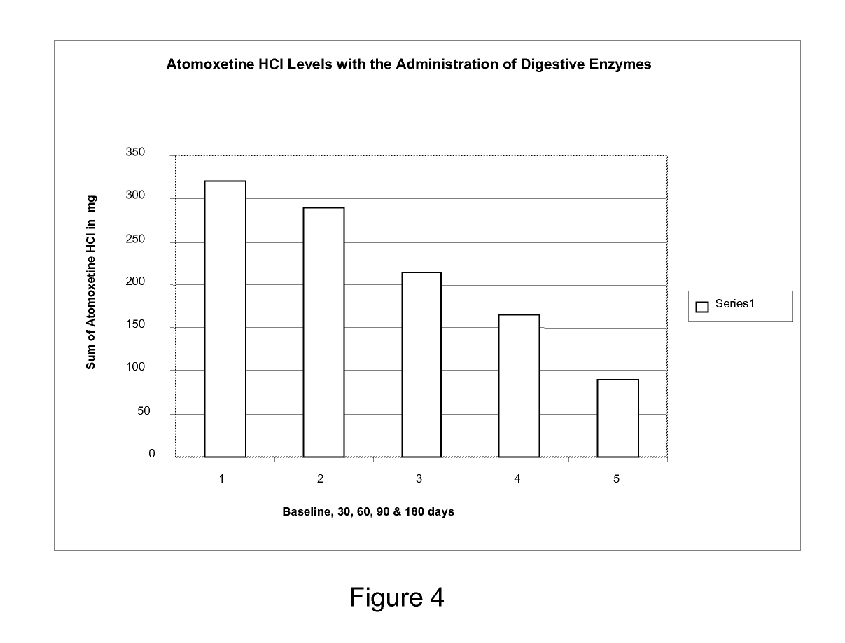

In a further study, 14 subjects diagnosed with ADD or ADHD and being treated with atomoxetine HCl were examined. Referring to FIG. 4, the subjects were taking a sum total of 320 mg of atomoxetine HCl prior to any treatment with the present invention. The subjects were treated with the present invention and monitored at 30, 60, 90 and 180 days. At 30 days, the sum total of methylphenidate being taken by the subjects had decreased to 290 mg. At 60 days, the sum total of methylphenidate being taken by the subjects had decreased to 215 mg. At 90 days, the sum total of methylphenidate being taken by the subjects had decreased to 165 mg. At 180 days, the sum total of methylphenidate being taken by the subjects had decreased to 90 mg.

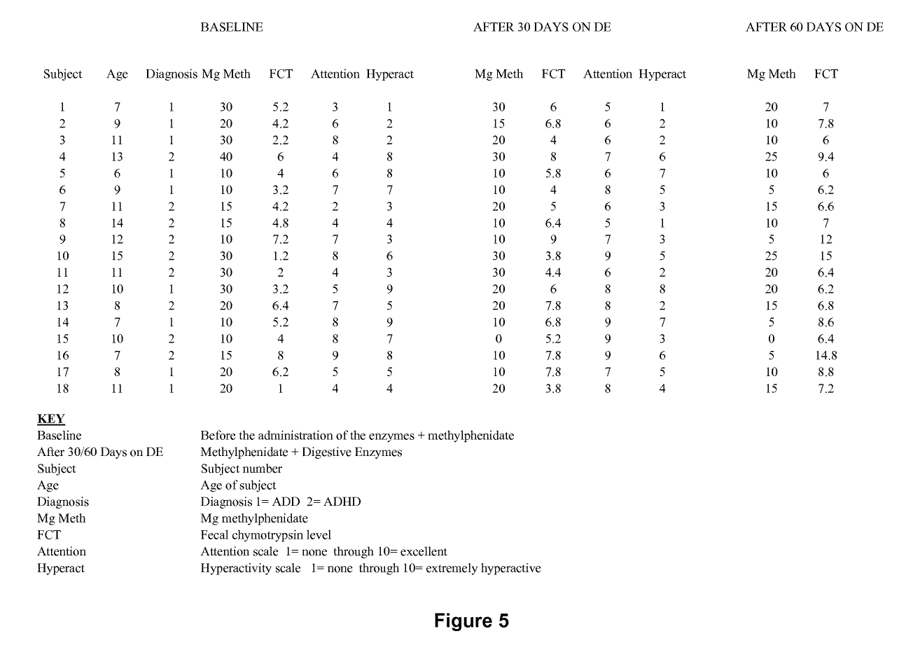

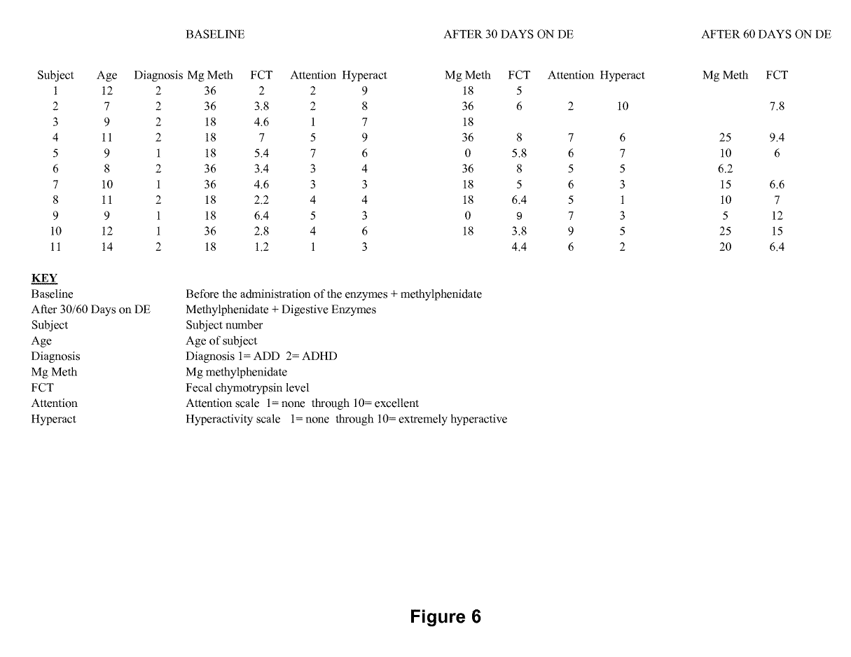

It has been shown that individuals taking the pharmaceutical preparation of the present invention have experienced a reduction in ADD/ADHD symptomology. As discussed in the preceding paragraphs, studies were conducted by the inventor in which individuals diagnosed with ADD or ADHD and being treated with methylphenidate (Ritalin®) and methylphenidate HCl (Concerta®) were examined. Physiological functions, such fecal chymotrypsin level were measured. Fecal chymotrypsin is a sensitive, specific measure of proteolytic activity. Decreased values suggest diminished pancreatic output (pancreatic insufficiency), hypoacidity of the stomach or cystic fibrosis. Elevated chymotrypsin values suggest rapid transit time, or less likely, a large output of chymotrypsin from the pancreas. Behavioral functions, such as attention span and hyperactivity levels were monitored. Referring to FIGS. 5 and 6, it can be seen that there was an increase in the fecal chymotrypsin levels and attention span levels, while there was a decrease in hyperactive behavior.

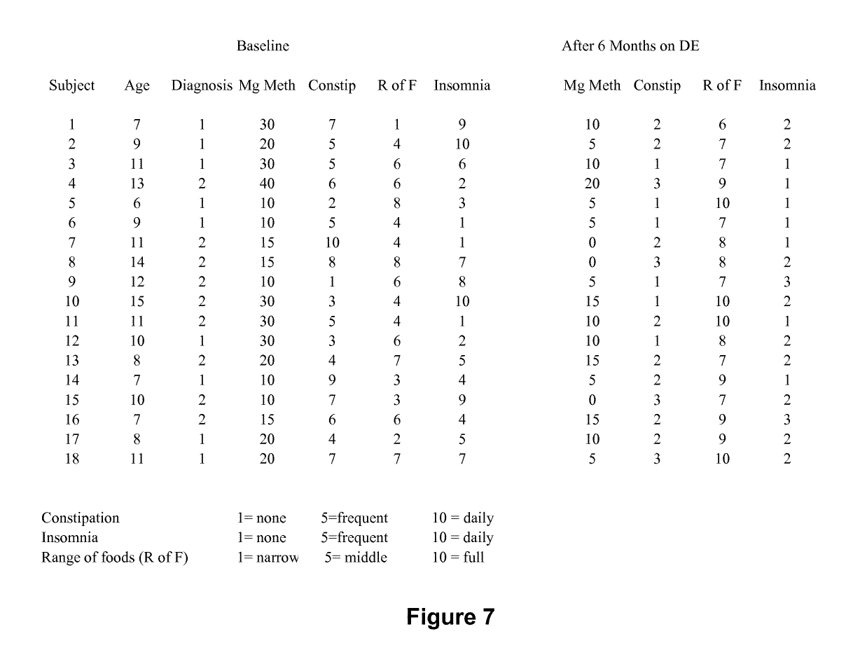

It has also been shown that individuals taking the pharmaceutical preparation of the present invention have experienced a reduction in the secondary symptoms of ADD/ADHD, such as constipation, decreased appetite, and insomnia. Referring to FIG. 7, it can be seen that there was a decrease in the instances of constipation and insomnia, while there was an increase in the range of foods that the subjects were able to consume. The results were similar for a group of 18 subjects being treated with methylphenidate hydrochloride (Concerta®).

In a further embodiment, the present invention is directed to a direct compression method for the manufacture of a pharmaceutical tablet preparation comprising the steps of: (a) forming an active blend by blending an intimate admixture of silicified microcrystalline cellulose and a therapeutic agent comprising one or more digestive enzymes; (b) forming a color blend by blending an intimate admixture of one or more pharmaceutically acceptable dyes and silicified microcrystalline cellulose if color is necessary; (c) combining the active blend, the color blend and a disintegrant into a preblend; (d) adding a lubricant to the preblend to form a final blend; and (e) compressing the final blend to form a pharmaceutical tablet preparation or a mixture of time released microtabs or a time released tablet.

This invention is accomplished by combining an ADD/ADHD medications (methylphenidate, methylphenidate HCl, Adderall®, atomoxetine HCl) and a therapeutically effective amount of digestive enzymes with one of the patented Prosolv technologies, i.e.: Prosolv SMCC 50 or Prosolv SMCC 90, or other Prosolv technologies. When employing the Prosolv method, the silicified microcrystalline cellulose (SMCC) used in the preparation of the present invention may be any commercially available combination of microcrystalline cellulose granulated with colloidal silicon dioxide. The SMCC generally will be as described in Sherwood et al, Pharm. Tech., October 1998, 78-88 and U.S. Pat. No. 5,585,115, which is incorporated herein by reference in its entirety. SMCC can be obtained commercially from Edward Mendell Company, Inc., a subsidiary of Penwest Ltd., under the name ProSolv SMCC. There are different grades of SMCC available, with particle size being the differentiating property among the grades. For example, ProSolv SMCC 90 has a median particle size, by sieve analysis, in the region of 90 micrometers. ProSolv SMCC 50 has a median particle size, by sieve analysis, in the region of about 40-50 micrometers.

The pharmaceutical preparation of the present invention may be prepared using a direct compression method, a dry granulation method, or by wet granulation. Preferably, the digestive/pancreatic enzyme preparation of the present invention will be prepared using a direct compression process. This preferred process consists of two main steps: blending and compression.

The blending step is composed of an active blend, color blend, pre-blend, and final blend (lubrication). The formulation of the present invention may include a number of other ingredients for optimal characteristics of the pharmaceutical composition. Such other ingredients and the amounts to be used are within the knowledge of persons having ordinary skill in the art and are known in the pharmaceutical arts. These may include disintegrates, lubricants and/or coloring agents among others. Suitable disintegrants include, for example, sodium starch glycolate, other starches such as pregelatinized starch, and celluloses. Suitable lubricants may be provided, such as magnesium stearate, calcium stearate, talc and stearic acid. Any coloring agent certified by the FDA may be used, such as FD&C Yellow #6, among others.

Whether utilizing the Prosolv method or other methodologies, such as enteric coating or lipid encapsulation, the pharmaceutical preparation of the present invention will be formulated and manufactured such that the particles will be uniformly distributed and there will be no overage with respect to the amount of enzyme found in the preparation. The new drug formulation can be found in, but is not limited to, formulations which include the ADD/ADHD medication and digestive/pancreatic enzymes with and without the utilization of the Prosolv technology.

The digestive/pancreatic enzyme combination component of the overall combination may include, but are not limited to, one or more of the following: amylases, proteases, cellulase, papaya, bromelain, lipases, chymotrypsin, and trypsin. These enzymes can be in the form of animal or plant derivatives, natural or synthetic.

Each of these combinations can be made into a pulse dose formulation wherein the time release portion of the tablet can be with the ADD/ADHD medication portion, the enzyme portion, or both. Therefore, dosing can be delivered in the tablet or micro-pellets in a single pulse delivery or a time release delivery. These combinations are not limited by number or scope of digestive enzymes or the dosing level of the ADD/ADHD medication. This invention is further unique by virtue of the compression and co-processing methodology which the Prosolv technology brings to the mixture of the ADD/ADHD medication and digestive enzyme. The pill size therefore can be significantly reduced, the amount of ADD/ADHD medication and digestive enzyme is significantly regulated and reproducible, and the combination can be delivered either directly through the pill and dissolved by the body, or can be delivered in a pulse dosing fashion which renders the digestive enzymes or its derivatives delivered in a time release fashion.

The Prosolv technology further adds improved material flow while maintaining compaction, manufacturing speeds can be improved, and allows for high or low drug loading applications as well as time or pulse release delivery. Further, the technology allows for a pill for tablet or micro tablet to be produced which has optimal content uniformity, direct compression without granulation, fewer numbers of excipients and fillers, and a smaller tablet.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

1-22. (canceled)

23. A method of treating Attention Deficit Disorder (ADD) or Attention Deficit Hyperactivity Disorder (ADHD) in a subject in need thereof, comprising administering to the subject a therapeutically effective amount of a first class of medication and a pharmaceutical preparation comprising digestive enzymes for at least six months, wherein the pharmaceutical preparation is effective to reduce the amount of the first class of medication used to treat ADD or ADHD in the subject; wherein the digestive enzymes comprise a protease, an amylase and a lipase, and wherein the first class of medication is selected from the group consisting of an amphetamine, an amphetamine salt, an atomoxetine HCl, and a combination thereof.

24. The method of claim 23, wherein the digestive enzymes further comprise a cellulase, a elastase, a hydrolase, or a combination thereof.

25. The method of claim 23, wherein the digestive enzymes are animal enzymes, plant enzymes, synthetic enzymes, or a combination thereof.

26. The method of claim 23, wherein the amount of the first class of medication used to treat ADD or ADHD ranges from about 1 mg to about 100 mg.

27. The method of claim 23, wherein the protease comprises chymotrypsin.

28. The method of claim 23, wherein the pharmaceutical preparation is a dosage formulation selected from the group consisting of a pill, a tablet, a capsule, a microcapsule, a mini-capsule, a time released capsule, a mini-tab, and a combination thereof.

29. The method of claim 23, wherein the pharmaceutical preparation is resistant to degradation by an environmental factor selected from the group consisting of light, heat, humidity, association with an excipient, and a combination thereof.

30. The method of claim 23, wherein the first class of medication is an extended-release medication.

31. The method of claim 23, wherein the pharmaceutical preparation comprises microcrystalline cellulose.

32. The method of claim 31, wherein the microcrystalline cellulose comprises silicified microcrystalline cellulose.

33. The method of claim 32, wherein the silicified microcrystalline cellulose has a median particle size of about 90 micrometers.

34. The method of claim 32, wherein the silicified microcrystalline cellulose has a median particle size of about 40-50 micrometers.

35. The method of claim 23, wherein the pharmaceutical preparation is formulated as sprinkles.

36. The method of claim 23, wherein the first class of medication is an amphetamine or a salt form thereof and is formulated in amount of from about 5 mg to about 100 mg.

37. The method of claim 23, wherein the first class of medication is atomexetine HCl and the subject is administered at a sum total of 320 mg atomexetine HCl prior to administration of the digestive enzymes.

38. The method of claim 23, wherein the first class of medication is atomexetine HCl and the subject is administered at a sum total of 290 mg of atomexetine HCl after administration of the medication and the digestive enzymes for about 30 days.

39. The method of claim 23, wherein the first class of medication is atomexetine HCl and the subject is administered at a sum total of 215 mg of atomexetine HCl after administration of the medication and the digestive enzymes for about 60 days.

40. The method of claim 23, wherein the first class of medication is atomexetine HCl and the subject is administered at a sum total of 165 mg of atomexetine HCl after administration of the medication and the digestive enzymes for about 90 days.

41. The method of claim 23, wherein the first class of medication is atomexetine HCl and the subject is administered at a sum total of 90 mg of atomexetine HCl after administration of the medication and the digestive enzymes for about 180 days.

42. The method of claim 23, wherein the first class of medication is an amphetamine or an amphetamine salt, and the subject is administered at a sum total of 2370 mg of the amphetamine or an amphetamine salt after administration of the medication and the digestive enzymes for about 30 days.

43. The method of claim 23, wherein the first class of medication is an amphetamine or an amphetamine salt, and the subject is administered at a sum total of 1720 mg of the amphetamine or an amphetamine salt after administration of the medication and the digestive enzymes for about 60 days.

44. The method of claim 23, wherein the first class of medication is an amphetamine or an amphetamine salt, and the subject is administered at a sum total of 1180 mg of the amphetamine or an amphetamine salt after administration of the medication and the digestive enzymes for about 90 days.

45. The method of claim 23, wherein the first class of medication is an amphetamine or an amphetamine salt, and the subject is administered at a sum total of 710 mg of the amphetamine or an amphetamine salt after administration of the medication and the digestive enzymes for about 180 days.

46. The method of claim 23, wherein the digestive enzymes are pancreatin.

47. A pharmaceutical preparation, comprising
(a) a therapeutically effective amount of a first class of medication used to treat Attention Deficit Disorder (ADD) or Attention Deficit Hyperactivity Disorder (ADHD); and
(b) digestive enzymes comprising protease, amylase and lipase.

48. A method for the manufacture of a pharmaceutical preparation by direct compression comprising the steps of: forming an active blend by blending an intimate admixture of silicified microcrystalline cellulose and a therapeutic agent comprising one or more digestive enzymes and one or more medications used to treat attention deficit disorder; forming a color blend by blending an intimate admixture of a plurality of pharmaceutically acceptable dyes and a silicified microcrystalline cellulose; combining the active blend, the color blend and a disintegrant into a preblend; adding a lubricant to the preblend to form a final blend; and compressing the final blend to form the pharmaceutical preparation.
